(19) United States
(12) Patent Application Publication
(10) Pub. No.: US 2024/0189495 A1
(43) Pub. Date: Jun. 13, 2024

(54) METHODS OF PREPARING AN EXTRACORPOREAL CIRCUIT FOR ANTICOAGULATION

(71) Applicant: Lawell Therapeutics, LLC, Hayward, CA (US)

(72) Inventors: James A. Wilkie, Dunstable, MA (US); Lakhmir Chawla, San Diego, CA (US)

(21) Appl. No.: 18/287,784

(22) PCT Filed: Apr. 8, 2022
(86) PCT No.: PCT/US22/23955

§ 371 Date: Oct. 20, 2023

Related U.S. Application Data
(60) Provisional application No. 63/177,164, filed on Apr. 20, 2021.

(51) Int. Cl. A61M 1/36

(52) U.S. Cl. CPC A61M 1/3672

(57) ABSTRACT

The present disclosure provides methods of preparing an extracorporeal circuit for circulating blood. The methods of preparation include ascertaining the adsorptive potential of an anticoagulant to a filter membrane material and adjusting priming of the circuit in accordance.

### RELATED APPLICATION

This application claims the benefit of priority to U.S. provisional application No. 63/177,164, filed Apr. 20, 2021, the contents of which are incorporated herein by reference in their entirety.

### BACKGROUND

Extracorporeal circuits, such as those used in renal replacement therapy (RRT), extracorporeal membrane oxygenation (ECMO), plasmapheresis, and cardiopulmonary bypass (CPB) can suffer from blood clotting within the circuit.

Because such an event can have severe consequences, there is a need for methods to prevent its occurrence.

### SUMMARY OF THE INVENTION

Methods are described herein to prepare an extracorporeal circuit for use. For example, in accord with the disclosed methods, for a given filter membrane in an extracorporeal circuit, certain priming parameters (e.g., duration, anticoagulant amount) are preferable over others.

The design and electronic charge of extracorporeal circuit membranes vary from one manufacturer to another as does the binding affinity of certain anticoagulants to the filter membrane. Some filter membranes will adsorb the anticoagulant to a great degree while others will not adsorb, or adsorb very little, of the anticoagulant. In some aspects, the quantity of the anticoagulant used to prime the circuit is based on its interaction with the filter membrane and the patient's tolerance for receiving an anticoagulant. In certain aspects, the user can first select the filter membrane, then select the anticoagulant, then select the priming quantity of an anticoagulant based on the filter membrane to be used and the patient's tolerance for anticoagulation.

### BRIEF DESCRIPTION OF THE FIGURES

FIG. 1: A schematic depicting a renal replacement therapy.

FIG. 2: A schematic of the regional anticoagulating effect of nafamostat, in which a coagulation parameter such as activated coagulation time (ACT) or activated partial thromboplastin time (aPTT) is used as a standard measure of anticoagulation.

FIG. 3A and FIG. 3B: An illustration of a sample vial that contains nafamostat (FIG. 3A) and an illustration of a chemical structure of nafamostat mesylate (FIG. 3B).

FIG. 4: A flowchart depicting an embodiment of a method for preparing an extracorporeal circuit connected to a subject's bloodstream.

FIG. 5: A schematic depicting an in vitro Dialysis Circuit.

### DETAILED DESCRIPTION OF THE INVENTION

Disclosed herein are methods for preparing an extracorporeal circuit for use. When the filter membrane is selected from the group of filters that will preferentially adsorb cytokines and other molecules within the bloodstream, the membrane may coincidentally adsorb the anticoagulant, leaving too little anticoagulant within the extracorporeal circuit to effectively anticoagulate a subsequent infusion of blood. In other instances, the anticoagulant may not be adsorbed onto the filter membrane and if too much anticoagulant is initially provided in the priming solution, then an overdose of anticoagulant will be inadvertently infused into the patient, which may be life threatening to a patient with a contraindication to receiving an anticoagulant. Thus, there is a need to properly match the anticoagulant to the extracorporeal filter membrane. The parameters can be selected by consulting a pre-compiled lookup table (e.g., directly through the values in the table, or based on the values in the table such as via interpolation or extrapolation).

### Definitions

As used in this specification, “a” and “an” can mean one or more. As used herein in the claim(s), when used in conjunction with the word “comprising,” the words “a” and “an” can mean one or more than one. As used herein, “another” can mean at least a second or more.

As used herein, “adsorb” or “adsorption” means the attachment by mechanical or chemical means of a molecule to a material.

As used herein, “prime,” “priming” or “prime solution” refers to the initial infusion of a solution into an extracorporeal circuit or catheter, whether or not the circuit is dry or previously filled with another solution or blood.

As used herein, “anticoagulating” includes preventing or reducing the coagulation of blood. For example, an agent anticoagulates blood if the blood has a longer clotting time in its presence as compared to in its absence.

As used herein, “without substantially affecting anticoagulation” refers to a change (or lack thereof) in a blood coagulation parameter such as activated partial thromboplastin time (aPTT), prothrombin time (PT), international normalized ratio (PT/INR), thromboelastography (TEG), or the activated coagulation time (ACT) that is at most 50% (e.g., −50%, −25%, −15%, −10%, −5%, 0%, +5%, +10%, +15%, +25%, +50%). For example, if the systemic ACT of a subject changes from 100 seconds to 110 seconds during a procedure, the procedure does not substantially affect anticoagulation in the subject, since the change in ACT is +10%, which is not more than 50%. The term “without substantially affecting anticoagulation” also refers to the systemic anticoagulation effect after a clinically acceptable time for allowing cessation of a systemic effect.

As used herein, “regional anticoagulation” refers to anticoagulation that is not systemic anticoagulation. It includes anticoagulating an extracorporeal circuit without substantially affecting anticoagulation within the systemic blood circulation of a subject whose bloodstream is connected to the extracorporeal circuit. Various coagulation parameter values can be used as a measure of regional anticoagulation, such as ACT, aPTT, or a combination thereof.

As used herein, “systemic anticoagulation” refers to anticoagulation within a subject's body, which, for example, can be measured from blood drawn directly from the patient. Various coagulation parameter values can be used as a measure of systemic anticoagulation, such as ACT, aPTT, or a combination thereof. In some embodiments, a therapeutically effective change in a coagulation parameter from blood drawn directly from the patient indicates a therapeutically effective systemic anticoagulation.

The term “subject” refers to a mammal, including, but not limited to, a human or non-human mammal, such as a bovine, equine, canine, ovine, or feline. In some embodiments, the subject is a human. In the specification, the term “patient” is used interchangeably with the term “subject.”

### Methods of Priming an Extracorporeal Circuit

Numerous types of blood purification therapies have been developed. Some patients require blood purification due to a lack of kidney function while other patients may benefit from blood purification even while their kidneys are functional.

Kidney dialysis is performed in patients with acute or chronic impairment in renal function. This type of blood purification focuses on a reduction in urea. Kidney dialysis may also be utilized to treat ingestions and/or poisoning. Many types of filter membranes exist in the marketplace and suppliers promote advantages of one over the other. Filter membrane materials sold in various parts of the world have been developed by companies with a focus on the local marketplace, therefore, there is no universally accepted membrane material that is utilized for kidney dialysis. Clinicians in different parts of the world have access only to certain types of membranes approved in the country by the corresponding regulatory agency and thus these clinicians do not have access to all type of membranes.

The adhesion of proteins to the membrane surface commonly occurs during treatment, and this new layer (‘protein cake’) acts as a barrier that further affects the transmembrane clearance by means of pore occlusion. This phenomenon leads to the so-called membrane fouling. Increasing the pore size of the membrane increases the time of onset and decreases the membrane fouling effect, allowing for prolonged clearance of molecules with lower (up to 20 kDa) molecular weight. Providing proper anticoagulation before and during use is known to minimize fouling and to prolong filter lifespan.

In some aspects, the filter membrane will adsorb the anticoagulant and the prime quantity of anticoagulant must be adjusted to provide adequate anticoagulation upon initiation of blood circulation. However, in other aspects, the filter membrane will not adsorb the anticoagulant and care must be taken to provide an adequate quantity of anticoagulant to initiate circulation of blood within the extracorporeal circuit but minimize the quantity of anticoagulant infused into the patient, especially if the patient has a contraindication for receiving anticoagulation. Improper selection of the anticoagulant and prime quantity of the anticoagulant can cause undue harm as the anticoagulant can be undesirably delivered to the patient.

In some aspects, the disclosure relates to priming the extracorporeal circuit without substantially affecting a subject's systemic coagulation function. Conditions that may require extra care when initiating extracorporeal circulation of blood and thus anticoagulation of the circuit include, but are not limited to, previous use of anticoagulants, recent cardiopulmonary bypass (CPB), gastrointestinal bleeding, persistent tracheal tube bleeding, and hemorrhagic cerebrovascular accident, in which prognoses that already are serious become worse, or life-threatening, if anticoagulants are administered to the subject. In general, a condition caused, at least in part, by internal bleeding (e.g., acute subarachnoid hemorrhage) can benefit from limiting further exposure of the subject to anticoagulation.

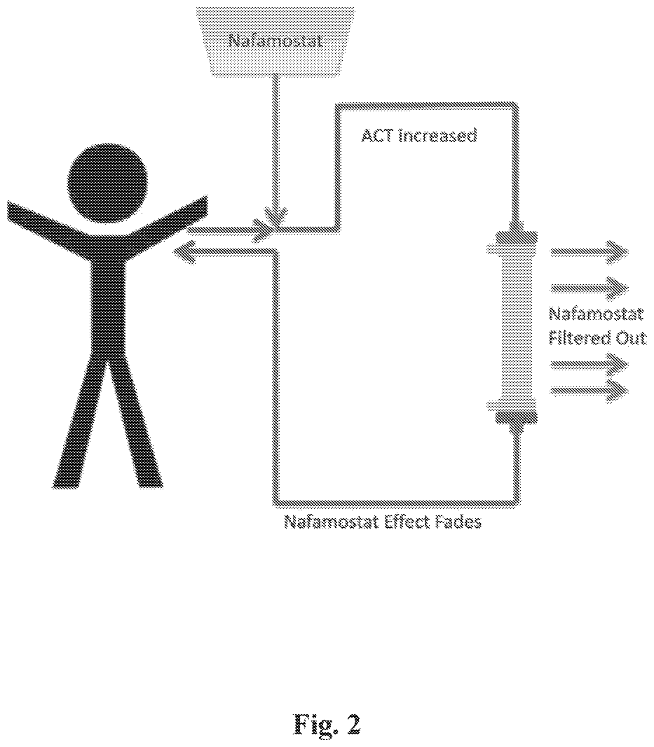

While anticoagulants in various membranes used for hemodialysis, ECMO, cytokine removal, cardiopulmonary bypass, and plasmapheresis have been used in the past, the field has failed to match the anticoagulant or priming dose with a particular membrane material to achieve a desired distribution of anticoagulant. The disclosed methods provide previously unattained benefits in being able to properly prescribe and subsequently control the regional circuit exposure to the anticoagulant separately from the patient's systemic exposure (FIG. 2).

In some embodiments, an extracorporeal circuit must be primed with an agent that prevents blood clotting. The priming volume and priming quantity of the anticoagulating agent is determined by choosing a filter membrane, ascertaining the absorptive characteristics of the anticoagulant to the membrane (which may be pre-determined), and determining the quantity with which to prime the circuit.

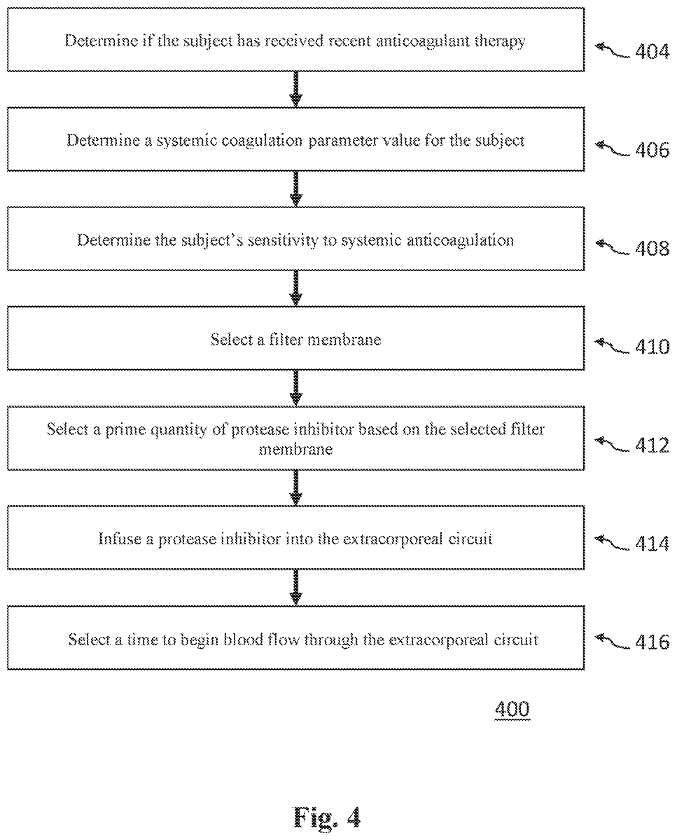

A particular exemplary embodiment of a method of priming an extracorporeal circuit is shown in FIG. 4 as method 400. Following through this outline of method 400, at block 404, one can determine if the subject has received anticoagulation therapy during a preceding 48-hour period. At block 406, one can determine a systemic coagulation parameter value for the subject's bloodstream, and at block 408 one can determine the subject's sensitivity to systemic anticoagulation. At block 410, one can select a filter membrane, which can be a polyacrylonitrile based membrane [such as AN69, AN69ST, oXiris], polysulfone, polyethersulfone, or polyarylethersulfone. At block 412, one can select a prime quantity of protease inhibitor based on the selected filter membrane. At block 414, one can infuse a priming quantity of an anticoagulating agent into the extracorporeal circuit at a location upstream of the filter in the extracorporeal circuit for the specified period of time. At block 416, one can select a time to begin blood flow through the extracorporeal circuit.

The priming steps further involve loading a certain quantity of anticoagulant into the filter such that when blood flow is initiated, the pre-existence of the anticoagulant will prevent clotting. The clinician must be cognizant of the fact that the residual quantity of anticoagulant that is unbound to the filter membrane will be circulated into the patient. If the patient has a contraindication to receiving a therapeutic dosage of an anticoagulant, then the quantity of anticoagulant must be carefully selected such that only a certain amount of anticoagulant is available for anticoagulation of blood in the filter and not for systemic anticoagulation. If such precautions are not undertaken, initiation of blood flow in the extracorporeal circuit may result in a rapid bolus of anticoagulant delivered to the patient, which may result in a significant change to systemic coagulation function.

Nafamostat is nafamostat free base. The concentration of nafamostat in nafamostat mesylate (nafamostat) is 63.2%. Therefore, 50 mg of nafamostat mesylate contains nafamostat 31.6 mg.

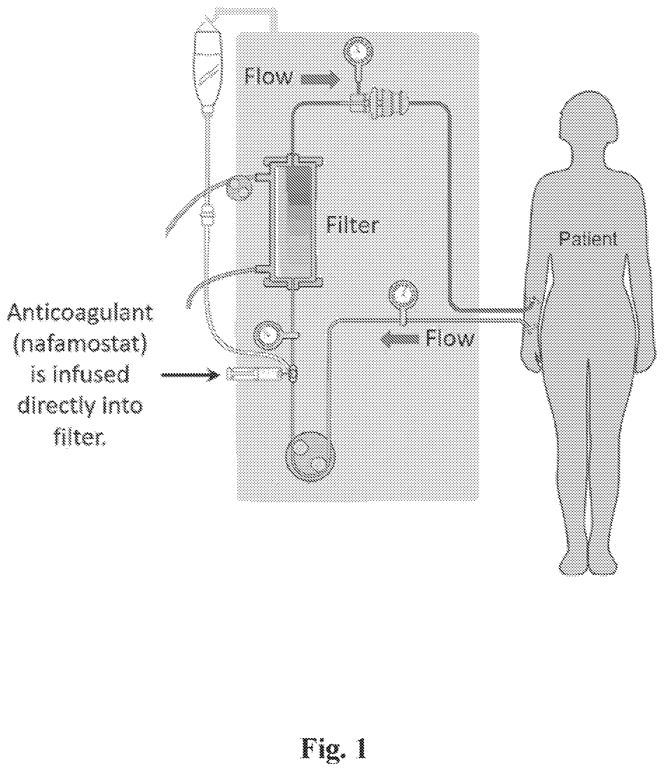

The are many different types of filter membranes used for renal replacement therapy (FIG. 1). The medical literature of clinical and non-clinical studies of nafamostat in Japan and South Korea report the use of surface treated polyacrylonitrile (AN69ST), polysulfone, polyarylethersulfone, but not polyacrylonitrile AN69 or polyethersulfone (PES), which are used in the United States.

In some embodiments, the adsorption characteristics of the anticoagulant to the filter membrane material must be known to choose the priming conditions. The priming conditions, in some embodiments, entail selecting an appropriate quantity of anticoagulant allowing the anticoagulant to circulate in the filter membrane but not in the presence of blood and ensuring that an appropriate quantity of anticoagulant remains available for satisfactory anticoagulation. The clinician, in some embodiments, must also know the quantity absorbed over a certain period of time such that blood flow is initiated within a certain timeframe before the quantity of anticoagulant placed into the prime solution is adsorbed by the filter.

The clinician, in some embodiments, must know the maximum time delay allowable after initiating priming of the filter and before initiating blood flow, otherwise, if the filter continuously adsorbs the anticoagulant, an insufficient quantity will remain.

The clinician, in some embodiments, must know to initiate the flow of anticoagulation prior to the flow of blood, especially in certain filters that exhibit high adsorption characteristics of the anticoagulant, otherwise the filter may clot prematurely.

### Nafamostat

In some aspects, the anticoagulant used by the disclosed methods is a protease inhibitor. In some aspects, the protease inhibitor is nafamostat, nafamostat mesylate, or another salt form of nafamostat.

For any embodiment disclosed herein in which a molecule (such as nafamostat) is used pharmacologically, the salt form can be a pharmaceutically acceptable salt form.

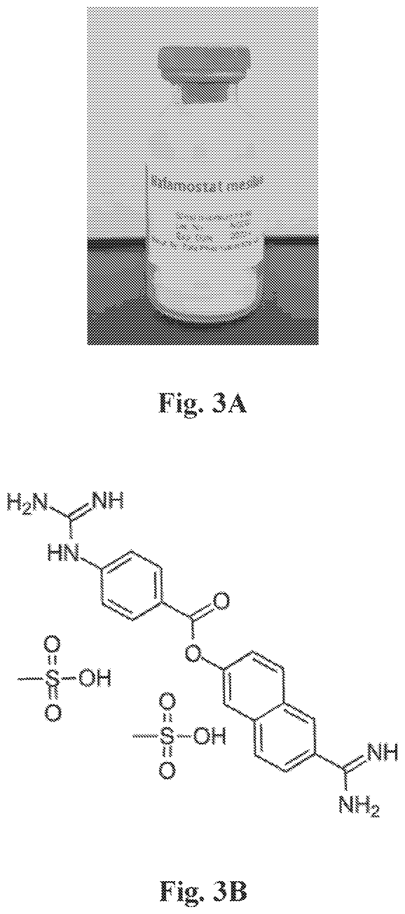

Nafamostat is a small molecule, broad spectrum, protease inhibitor that inhibits thrombin at the platelet thrombin receptor, PAR1. Because of nafamostat's potential adsorption to some membranes, only a limited amount may reach a subject. Nafamostat can, in some embodiments, be infused as part of a composition, such as the one described in the Examples. Nafamostat can be produced as part of a product vial, an example of which is shown in FIG. 3A. A representative illustration of the chemical structure of nafamostat mesylate is shown in FIG. 3B.

### EXAMPLES

### Example 1

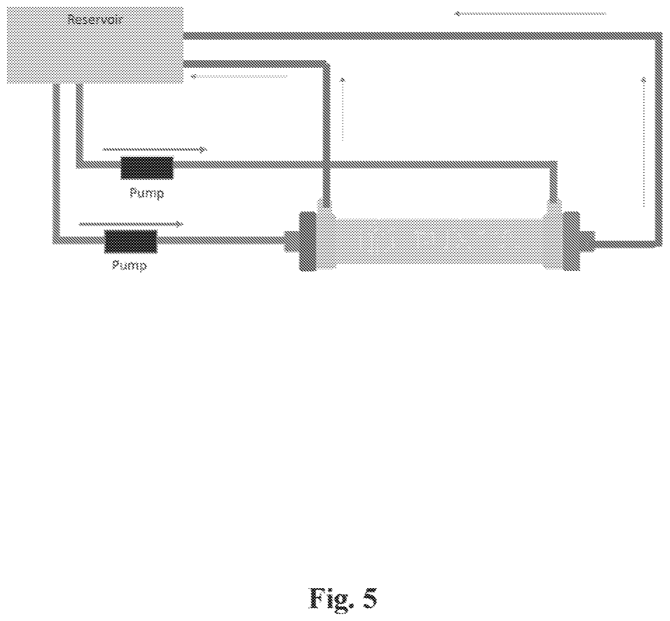

A dialysis apparatus was established as described in FIG. 5 without using a filter. An experiment was conducted to determine the adsorption characteristics to the tubing. The solution was circulated through the tubing at typical CRRT flow rates of 100-300 milliliters per minute (FIG. 5). The concentration of nafamostat was measured via HPLC at 0, 5, 10 and 20 minutes. Only one reservoir of nafamostat was used. Therefore, any change in concentration represents adsorption since the molecule cannot escape the system.

As presented in Table 1, nafamostat does not adsorb substantially onto the tubing used for all tests. Therefore, any change in concentration can be attributed to adsorption onto the filter membrane tested.

TABLE 1
Percent of Nafamostat Remaining as a Ratio
of the Starting Concentration at Time 0.

| Time (min) | Tubing |
|---|---|
| 0 | 100% |
| 5 | 100% |
| 10 | 101% |
| 20 | 100% |

### Examples 2 Through 7

A dialysis apparatus was established as described in FIG. 5 using a PS, PES, PAES, or a PAN filter. A series of experiments was conducted using various filter membranes in an in vitro model to determine the filter membrane adsorption characteristics. A solution of nafamostat was circulated through the blood and dialysate sides of the filter at typical CRRT flow rates of 100-300 milliliters per minute. The concentration of nafamostat was measured via HPLC at 0, 5, 10 and 20 minutes. Only one reservoir of nafamostat was used. Therefore, any change in concentration represents adsorption since the molecule cannot escape the system. The following filters were used: Polyethersulfone (PES) from NxStage, part number CAR-505; Polyarylethersulfone (PAES) from Baxter, part number HF1000; Polyacrylonitrile (PAN) from Baxter, part number M100; Polysulfone (PS) from Baxter, part number F50; no filter, tubing only from Baxter, part number M100. Baxter tubing was used for all filter tests.

TABLE 2
Prime Quantity and Time Determination

| | | Example Number | | | |
|---|---|---|---|---|---|
| | | 2 | 3 | 4 | 5 |
| | | Filter material | | | |
| | | PS | PES | PAES | PAN |
| | | Manufacturer's part number | | | |
| | | F50 | CAR-505 | HF1000 | M100 |
| Nafamostat free base concentration | mcg/mL | 158 | 158 | 158 | 158 |
| Solution volume | mL | 500 | 500 | 500 | 500 |
| Prime Quantity | mcg | 79,000 | 79,000 | 79,000 | 79,000 |
| | | Quantity (% nafamostat free base) remaining | | | |
| Time after initiation of priming (minutes) | | after the initiation of priming | | | |
| 0 | | 100% | 100% | 100% | 98.7% |
| 5 | | 96.7% | 95.6% | 96.9% | 20.5% |
| 10 | | 96.8% | 95.6% | 97.2% | 4.3% |
| 20 | | 97.2% | 95.6% | 97.1% | 0.3% |
| | | Quantity (mcg nafamostat free base) adsorbed | | | |
| Time after initiation of priming (minutes) | | over time (minutes) | | | |
| | 5 | 2,620 | 3,555 | 2,446 | 61,770 |
| | 10 | 2,559 | 3,555 | 2,184 | 74,594 |
| | 20 | 2,222 | 3,555 | 2,261 | 77,732 |
| | Avg. | 2,467 | 3,555 | 2,297 | 71,365 |
| Prime quantity for clinical practice | mcg | 3,000 | 4,000 | 3,000 | 63,200 |
| (mcg nafamostat free base) | | | | | |
| Prime quantity for clinical practice | mg | 3 | 4 | 3 | 63.2 |
| Prime volume | mL | 500 | 500 | 500 | 500 |
| Priming time | minutes | 5 | 5 | 5 | 10 |
| Unacceptable delay in starting infusion of blood | minutes | >20 | >20 | >20 | >5 |

PS: polysulfone; PES, polyethersulfone; PAES: polyarylethersulfone; PAN: polyacrylonitrile.

TABLE 3
Prime Quantity and Time Determination

| | | Example | | |
|---|---|---|---|---|
| | | 5 | 6 | 7 |
| | | Filter material | | |
| | | PAN | PAN | PAN |
| | | Manufacturer's Part Number | | |
| | | M100 | M100 | M100 |
| Nafamostat free base concentration | mcg/mL | 158 | 316 | 632 |
| Solution volume | mL | 500 | 500 | 500 |
| Prime Quantity | mcg | 79,000 | 158,000 | 316,000 |
| | | Quantity (% nafamostat free base) remaining | | |
| Time after initiation of priming (minutes) | | after the initiation of priming | | |
| | 0 | 98.7% | 99.5% | 99.1% |
| | 5 | 20.5% | 20.4% | 17.4% |
| | 10 | 4.3% | 4.3% | 2.7% |
| | 20 | 0.3% | 0.4% | 0.04% |
| | | Quantity (mcg nafamostat free base) adsorbed | | |
| Time after initiation of priming (minutes) | | over time (minutes) | | |
| | 5 | 61,770 | 124,946 | 258,314 |
| | 10 | 74,594 | 150,288 | 304,629 |
| | 20 | 77,732 | 156,502 | 313,065 |
| Avg. | | 71,365 | 143,912 | 292,003 |
| Prime Quantity for Clinical Practice | mcg | 31,600 | 31,600 | 31,600 |
| Prime Quantity for Clinical Practice | mg | 31.6 | 31.6 | 31.6 |
| Prime Volume | mL | 500 | 500 | 500 |
| Unacceptable delay in starting infusion of blood | minutes | >5 | >5 | >5 |

PAN: polyacrylonitrile.

Table 3 shows that the quantity of nafamostat adsorbed onto PAN filter is far greater than that adsorbed onto the non-PAN filters. The change in concentration in the non-PAN filters is complete within the first 5 minutes regardless the solution concentration (i.e., the filter absorbs what it is going to absorb in 5 minutes). The change in concentration in the PAN filters is only 80% within the first 5 minutes, 95% within the first 10 minutes, 99% within the first 15 minutes, and 99.5% within the first 20 minutes.

### Example 8

To determine if a polyacrylonitrile filter can be saturated, and if so, what quantity of nafamostat would be required, an in vitro experiment was conducted wherein 632 milligrams of nafamostat were passed through the filter in the same manner as described in FIG. 5 for 20 minutes. Then the solution was replenished with an additional 632 milligrams (solution B-1), which was circulated for an additional 20 minutes, followed by replenishment with an additional 632 milligrams (solution B-2), which was circulated for an additional 20 minutes and finally replenished with an additional 379 milligrams. The total quantity of 2275 milligrams represents a three-day consumption of nafamostat at the maximum dose of 31.2 mg/h. As one can see from Table 4, nafamostat continues to adsorb to the polyacrylonitrile filter even after the entire quantity of 2275 milligrams has been delivered.

TABLE 4
Amount of Nafamostat Remaining as a
Ratio of the Starting Concentration at Time 0.
Example 8

| Time | Solution A | Solution B-1 | Solution B-2 | Solution B-3 |
|---|---|---|---|---|
| (min) | 632 mg | Added 632 mg | Added 632 mg | Added 379 mg |
| 0 | 99.2% | 100.0% | 100.0% | 100.0% |
| 5 | 8.5% | 14.6% | 20.0% | 20.8% |
| 10 | 0.8% | 6.2% | 14.6% | 19.2% |
| 20 | 0.0% | 3.1% | 10.8% | 17.7% |

Table 4 shows that if the priming solution is replenished with additional quantities of nafamostat, the filter will continue to absorb molecule. Thus, the PAN filter cannot be saturated with nafamostat, and care must be taken to first initiate infusion of a saline solution or heparinized saline solution to prime circuit, then initiate infusion of a nafamostat solution immediately prior to initiation of blood flow through the filter.

Above prime quantities are based on one square meter surface area and should be adjusted linearly for any change in surface area of the filter. The residual quantity of nafamostat will not provide an overdose when the extracorporeal circuit is started.

The priming amount and anticoagulant remaining in the circuit is important for both patient safety and circuit efficacy. If a large amount of anticoagulant is utilized in the priming sequence and only a small proportion is adsorbed by the filter, then the remaining anticoagulant will be flushed into the patient when the dialysis treatment is started, resulting in a bolus dose of anticoagulant, which can be dangerous for the patient. However, if insufficient anticoagulant is in the priming system, then the circuit may clot when the treatment starts as there will be insufficient anticoagulant in the priming solution to prevent clotting.

### Conditions and Procedures

In some aspects, the methods disclosed herein are applicable during certain procedures and for certain conditions.

When using a filter with a high adsorptive potential, such as PAN membrane, the user must pay particular attention to the delay between the period of priming and the onset of blood flow into the filter. Otherwise, the anticoagulated priming solution could be depleted of anticoagulant. If the quantity of anticoagulant remaining in solution after 5 minutes of priming is above 90%, as is the case with PS, PES, and PAES, then the filter is deemed to have a low adsorptive potential. The delay in the onset of blood flow from the period after priming can be longer with a filter that has a low adsorptive potential than the delay in the onset of blood flow from the period after the five minute priming with a filter that has a high adsorptive potential because less anticoagulant is adsorbed onto the filter within the first 5 minutes.

TABLE 5
Instructions for Priming

| Filter Material | | | | |
|---|---|---|---|---|
| (adsorptive | Priming | Priming | | |
| assessment) | Time | Amount | | Key Instructions |
| Non-PAN | 5-40 | 2-5 | mg | Start the anticoagulant pump 0 |
| | minutes | | | to 5 minutes prior to initiation |
| | | | | of blood flow. |
| PAN | Less than | 30 | mg | Start the anticoagulant pump 0 |
| | 5 min | | | to 5 minutes prior to initiation |
| | | | | of blood flow. |
| PAN | 5-40 | 0 | mg | Start the anticoagulant pump |
| | minutes | | | not less than 5 minutes prior to |
| | | | | initiation of blood flow. |

### INCORPORATION BY REFERENCE

Each publication and patent mentioned herein is hereby incorporated by reference in its entirety. In case of conflict, the present application, including any definitions herein, will control.

### EQUIVALENTS

While specific embodiments of the subject invention have been discussed, the outside specification is illustrative and not restrictive. Many variations of the invention will become apparent to those skilled in the art upon review of this specification and the following claims. The full scope of the invention should be determined by reference to the claims, along with their full scope of equivalents, and the specification, along with such variations.

What is claimed is:

1. A method of preparing an extracorporeal circuit for blood flow through the circuit, the method comprising
ascertaining the adsorptive potential of an anticoagulant to a filter membrane material within the extracorporeal circuit;
determining a priming quantity of the anticoagulant for the extracorporeal circuit based on the adsorptive potential;
determining a duration of system priming; and
infusing the priming quantity of the anticoagulant into the extracorporeal circuit for the selected duration of system priming.

2. The method of claim 1, wherein the anticoagulant is also a protease inhibitor or a thrombin inhibitor.

3. The method of claim 1, wherein the adsorptive potential is ascertained to be low.

4. The method of claim 3, wherein the filter membrane material comprises polysulfone (PS), polyethersulfone (PES), or polyarylethersulfone (PAES).

5. The method of claim 3, wherein the priming quantity is 2 mg to 5 mg.

6. (canceled)

7. The method of claim 3, wherein said duration of system priming is 5 mins to 30 mins.

8. (canceled)

9. The method of claim 3, further comprising initiating blood flow in the extracorporeal circuit before the expiration of the priming effect; no later than 2, 3, 4, 5, 6, 7, 8, 9, or 10 mins after said duration of system priming; or after initiating a post-priming infusion of the anticoagulant into the extracorporeal circuit.

10-12. (canceled)

13. The method of claim 1, wherein the adsorptive potential is ascertained to be high.

14. The method of claim 13, wherein the filter membrane material comprises polyacrylonitrile (PAN).

15. The method of claim 13, wherein the priming quantity is 2 mg to 40 mg.

16. (canceled)

17. The method of claim 13, wherein said duration is 2 mins to 5 mins.

18. (canceled)

19. The method of claim 13, further comprising initiating blood flow in the extracorporeal circuit before the expiration of the priming effect; no later than 2, 3, 4, or 5 mins after said duration of priming; or after initiating a post-priming infusion of the anticoagulant into the extracorporeal circuit.

20-22. (canceled)

23. A method of preparing an extracorporeal circuit for blood flow through the circuit, the method comprising
ascertaining the adsorptive potential of an anticoagulant to a filter membrane material within the extracorporeal circuit;
determining a duration of system priming; and
infusing a priming solution into the extracorporeal circuit for the selected duration of system priming.

24. The method of claim 23, wherein the anticoagulant is also a protease inhibitor or a thrombin inhibitor.

25. The method of claim 23, wherein the priming solution comprises anticoagulant-free saline and/or a priming quantity of the anticoagulant.

26. The method of claim 23, wherein the adsorptive potential is ascertained to be high.

27. The method of claim 26, wherein the filter membrane material comprises polyacrylonitrile (PAN).

28. The method of claim 26, wherein said duration is 2 mins to 5 mins.

29. (canceled)

30. The method of claim 26, further comprising initiating blood flow in the extracorporeal circuit, wherein blood flow is initiated not later than 5 mins after said duration of system priming.

31-32. (canceled)

33. The method of claim 26, further comprising initiating blood flow in the extracorporeal circuit after initiating post-priming infusion of said anticoagulant into the extracorporeal circuit.

34. The method of claim 1, further comprising adjusting the prime quantity of the anticoagulant based on filter membrane surface area, wherein said adjusting is linearly proportional.

35-37. (canceled)

38. The method of claim 2, wherein the anticoagulant is also a protease inhibitor or a thrombin inhibitor selected from nafamostat, nafamostat mesylate, or another salt form of nafamostat, abexate, gabexate mesylate, or another salt form of gabexate, hirudin, bivalirudin, desirudin, lepirudin, argatroban, dabigatran, efegatran, inogatran, melagatran, nafamostat, ximelagatran, gabexate, camostat, or another salt form thereof.

39. The method of claim 38, wherein the anticoagulant is nafamostat mesylate and the priming quantity is with respect to nafamostat free-base.

40-42. (canceled)

43. The method of claim 24, wherein the anticoagulant is also a protease inhibitor or a thrombin inhibitor selected from nafamostat, nafamostat mesylate, or another salt form of nafamostat, abexate, gabexate mesylate, or another salt form of gabexate, hirudin, bivalirudin, desirudin, lepirudin, argatroban, dabigatran, efegatran, inogatran, melagatran, nafamostat, ximelagatran, gabexate, camostat, or another salt form thereof.

44. The method of claim 43, wherein the anticoagulant is nafamostat mesylate and the priming quantity is with respect to nafamostat free-base.
